(19) United States
(12) Patent Application Publication
(10) Pub. No.: US 2025/0109050 A1
(43) Pub. Date: Apr. 3, 2025

(54) WATER TREATMENT SYSTEM, AERATION AMOUNT CONTROL DEVICE, AND AERATION AMOUNT CONTROL METHOD

(71) Applicant: Mitsubishi Electric Corporation, Tokyo (JP)

(72) Inventors: Wataru YOSHIDA, Tokyo (JP); Yoshifumi HAYASHI, Tokyo (JP); Eiji IMAMURA, Tokyo (JP); Seiji NODA, Tokyo (JP); Kenta SHIMODA, Tokyo (JP)

(73) Assignee: Mitsubishi Electric Corporation, Tokyo (JP)

(21) Appl. No.: 18/844,061

(22) PCT Filed: Mar. 14, 2022
(86) PCT No.: PCT/JP2022/011233

§ 371 Date: Sep. 5, 2024

(51) Int. Cl. C02F 3/00; C02F 3/12; G01N 33/18

(52) U.S. Cl. CPC C02F 3/006; C02F 3/1278; G01N 33/18; C02F 2209/001; C02F 2209/38

(57) ABSTRACT

A water treatment system includes: a first water quality measurement value acquisition unit which acquires a water quality measurement value at a first time point, of treatment target water; a water quality variation pattern acquisition unit which, acquires a plurality of water quality variation patterns; an inflow water quality estimation unit which selects a water quality variation pattern that matches an acquisition condition for the water quality measurement value at the first time point, from the water quality variation patterns in the water quality variation pattern acquisition unit, and estimates an inflow water quality value subsequent to the first time point from the selected water quality variation pattern; and a control unit which controls an aeration amount from a blower to a reaction tank subsequent to the first time point, on the basis of the estimated inflow water quality value.

### TECHNICAL FIELD

The present disclosure relates to a water treatment system, an aeration amount control device, and an aeration amount control method.

### BACKGROUND ART

One of treatment methods for sewage containing organic substances and nitrogen is an activated sludge process. In the activated sludge process, microorganisms (activated sludge) having a purification ability are stored in a reaction tank and the microorganisms and waste water are mixed and contact with each other while being aerated, whereby contaminants in the waste water are oxidized and decomposed. In order to sufficiently purify the contaminants, it is necessary to supply (aerate) an appropriate amount of air to the bioreactor tank. In addition, the residence time in the reaction tank is as long as about 10 hours, and therefore it is necessary to control the aeration amount in accordance with variation in the inflow water quality (contaminant concentration in inflow water).

In this regard, it is known that aeration amount control is performed using an inflow water quality acquired through measurement by a sensor capable of continuously measuring the inflow water quality or through estimation from the past time-series data (see, for example, Patent Documents 1 and 2).

### CITATION LIST

### Patent Document

Patent Document 1: Japanese Patent No. 6764487
Patent Document 2: Japanese Laid-Open Patent Publication No. 2005-125229

### SUMMARY OF THE INVENTION

### Problem to be Solved by the Invention

In Patent Document 1, in order to continuously measure the water quality of inflow water, a water quality sensor such as an ammonia meter, a total nitrogen analyzer, or a BOD meter is provided, whereby high-accuracy measurement is achieved. However, these measurement instruments are expensive and need to be calibrated and maintained highly frequently, so that a lot of cost and effort are required for sensor maintenance.

In Patent Document 2, in determining a target value in water quality control, the inflow water quality is predicted on the basis of the past time-series data. However, in a case where the measured inflow water quality at present has changed from the water quality at the time when the past data was acquired, prediction accuracy might be lowered.

The present disclosure has been made to solve the above problem, and an object of the present disclosure is to provide a water treatment system, an aeration amount control device, and an aeration amount control method that can estimate an inflow water quality with high accuracy without using multiple measurement instruments for continuously measuring the inflow water quality, and supply a necessary amount of aeration without delay in accordance with variation in the inflow water quality, thus suppressing variation in a treated water quality.

### Means to Solve the Problem

A water treatment system according to the present disclosure is a water treatment system for performing water treatment through biological oxidation while performing aeration from a blower to a reaction tank, the water treatment system including: a first water quality measurement value acquisition unit which acquires a water quality measurement value at a first time point, of treatment target water flowing into the reaction tank; a water quality variation pattern acquisition unit which, from time-series change in water quality information of the treatment target water acquired in advance, acquires a water quality variation pattern according to a condition at a time of acquisition of the water quality information acquired in advance; an inflow water quality estimation unit which selects a water quality variation pattern that matches a condition at a time of acquisition of the water quality measurement value at the first time point, from the water quality variation patterns included in the water quality variation pattern acquisition unit, and estimates an inflow water quality value subsequent to the first time point from the selected water quality variation pattern; and a control unit which controls an aeration amount of the blower subsequent to the first time point, on the basis of the inflow water quality value estimated by the inflow water quality estimation unit.

### Effect of the Invention

The water treatment system according to the present disclosure can estimate an inflow water quality value with high accuracy without using a measurement instrument capable of continuously measuring the inflow water quality and supply a necessary amount of aeration in accordance with variation in the inflow water quality value, thus suppressing variation in a treated water quality value and reducing excessive aeration.

### BRIEF DESCRIPTION OF THE DRAWINGS

FIG. 1 is a block diagram showing the configuration of a water treatment system according to embodiment 1.

FIG. 2 is a block diagram showing the aeration amount control device according to embodiment 1.

FIG. 3 illustrates an inflow water quality variation pattern according to embodiment 1.

FIG. 4 shows an example of a water quality variation pattern selected by the aeration amount control device according to embodiment 1.

FIG. 5 shows an operation flow of the aeration amount control device according to embodiment 1.

FIG. 6 is a block diagram showing an aeration amount control device according to embodiment 2.

FIG. 7 shows an example of a water quality variation pattern selected by the aeration amount control device according to embodiment 2.

FIG. 8 is a block diagram showing the configuration of a water treatment system according to embodiment 3.

FIG. 9 is a hardware configuration diagram of an inflow water quality estimation unit and the aeration amount control device according to each of embodiments 1 to 3.

FIG. 10 shows the configuration of an inflow water quality inference device according to embodiment 4.

FIG. 11 shows the configuration of a learning device of the inflow water quality inference device according to embodiment 4.

FIG. 12 is a flowchart for performing learning using the learning device shown in FIG. 11.

FIG. 13 shows the configuration of an inference device according to embodiment 4.

FIG. 14 is a flowchart for inferring an inflow water quality value using the inference device shown in FIG. 13.

### DESCRIPTION OF EMBODIMENTS

Hereinafter, embodiments will be described with reference to the drawings. In the drawings, the same reference characters denote the same or corresponding parts. Hereinafter, with reference to the drawings, embodiments of the present disclosure will be described in detail. In the description and the drawings, components having substantially the same function are denoted by the same reference characters and will not repeatedly be described. In the drawings, illustration of configurations of devices and shapes of parts merely represent schematic configurations and shapes of the devices and the parts, Relative sizes and relative positions of parts shown in the drawings do not necessarily represent accurate size relationships and accurate positional relationships between the actual parts.

### Embodiment 1

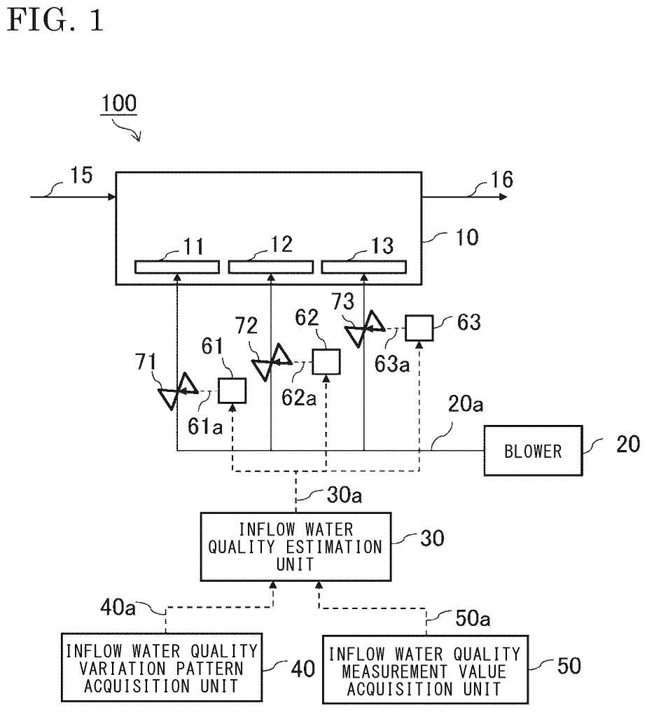

Hereinafter, a water treatment system according to embodiment 1 will be described with reference to FIG. 1 to FIG. 5.

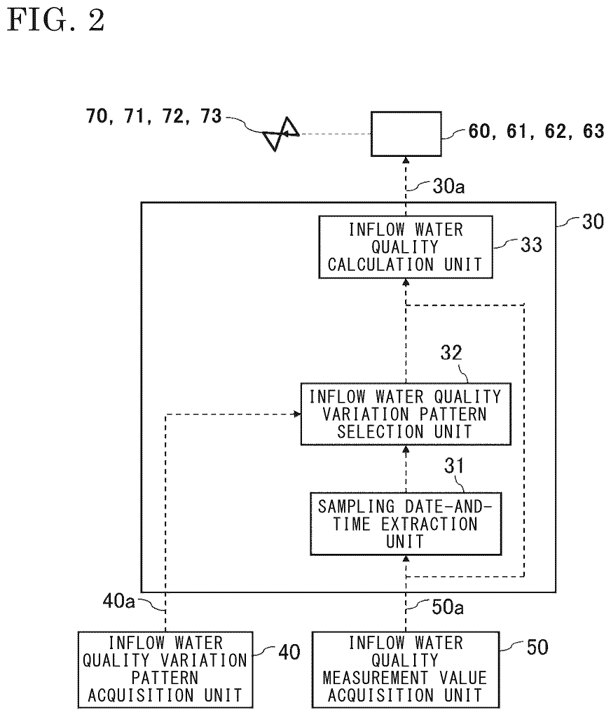

FIG. 1 shows the configuration of the water treatment system according to embodiment 1, and FIG. 2 shows the configuration of an aeration amount control device. In FIG. 1, a water treatment system 100 includes: a bioreactor tank 10 provided with an inflow section 15 for introducing treatment target water, an outflow section 16 for discharging treated water, and diffuser plates 11, 12, 13 disposed inside; a blower 20 which sends air to the diffuser plates 11, 12, 13; air volume adjustment valves 71, 72, 73 for adjusting the volume of air from the blower 20; target aeration amount calculation units 61, 62, 63 which calculate aeration amounts for controlling the air volume adjustment valves 71, 72, 73; an inflow water quality estimation unit 30 which estimates an inflow water quality value for the target aeration amount calculation units 61, 62, 63 to calculate the aeration amounts; an inflow water quality measurement value acquisition unit 50 which acquires a measurement value of a water quality of the treatment target water flowing into the bioreactor tank 10 and transmits the measurement value to the inflow water quality estimation unit 30; and an inflow water quality variation pattern acquisition unit 40 which acquires a variation pattern of an inflow water quality and transmits the variation pattern to the inflow water quality estimation unit 30.

The aeration amount control device is composed of air volume adjustment valves 70 (71, 72, 73), target aeration amount calculation units 60 (61, 62, 63), the inflow water quality estimation unit 30, the inflow water quality measurement value acquisition unit 50, and the inflow water quality variation pattern acquisition unit 40. When the air volume adjustment valves and the target aeration amount calculation units are collectively mentioned, they are respectively referred to as air volume adjustment valves 70 and target aeration amount calculation units 60, and when they are mentioned individually, they are respectively referred to as air volume adjustment valves 71, 72, 73 and target aeration amount calculation units 61, 62, 63.

Hereinafter, the details of each component will be described.

The bioreactor tank 10 is a reaction tank storing activated sludge therein. Air supplied from the blower 20 passes through a pipe 20a and is supplied into the bioreactor tank 10 from the diffuser plates 11, 12, 13.

The air volume adjustment valves 71, 72, 73 are provided one by one to pipes branched from the pipe 20a to the diffuser plates 11, 12, 13, respectively. Through adjustment of the opening degrees of the air volume adjustment valves 71, 72, 73, the amounts of aeration to be supplied to the diffuser plates 11, 12, 13 can be individually adjusted.

The target aeration amount calculation units 61, 62, 63 calculate target values for the amounts of aeration to be supplied from the diffuser plates 11, 12, 13, in every arbitrary cycle, and transmit the target values for the aeration amounts to the air volume adjustment valves 71, 72, 73 via signal lines 61a, 62a, 63a. The cycle of calculation of the target values for the aeration amounts is desirably about 1 second to 5 minutes, but may be arbitrarily set in accordance with the characteristic of the plant site. The opening degrees of the air volume adjustment valves 71, 72, 73 are adjusted so that the amounts of aeration supplied from the diffuser plates 11, 12, 13 become equal to the target values for the aeration amounts calculated by the target aeration amount calculation units 61, 62, 63. In FIG. 1, the example in which three diffuser plates, three air volume adjustment valves, and three target aeration amount calculation units are provided is shown, but the numbers thereof are not limited thereto. The numbers of the diffuser plates, the air volume adjustment valves, and the target aeration amount calculation units can be arbitrarily changed in accordance with the scale of the bioreactor tank 10 and the characteristic of the plant site,

The inflow section 15 is a pipe or a water channel through which treatment target water flows into the bioreactor tank 10. The outflow section 16 is a pipe or a water channel through which treated water treated in the bioreactor tank 10 flows to the outside of the bioreactor tank 10. The treatment target water flowing from the inflow section 15 comes into contact with activated sludge and air supplied through aeration in the bioreactor tank 10, whereby oxidation and decomposition of contaminants in the treatment target water are promoted, and the resultant water flows out as the treated water.

Next, the inflow water quality variation pattern acquisition unit 40 will be described.

A pattern of contaminant concentration variation over time in which contaminant concentrations in treatment target water acquired in advance are arranged in time series in accordance with the acquisition times, is inputted to and stored in the inflow water quality variation pattern acquisition unit 40. As the contaminants, one or more kinds are selected from treatment targets in the bioreactor tank 10. Examples include biochemical oxygen demand (BOD), chemical oxygen demand (COD), ammonia nitrogen, total nitrogen, Kjeldahl nitrogen, total phosphorus, and phosphate phosphorus. A plurality of inflow water quality variation patterns can be inputted in accordance with the kinds of contaminants or a feature of date and time.

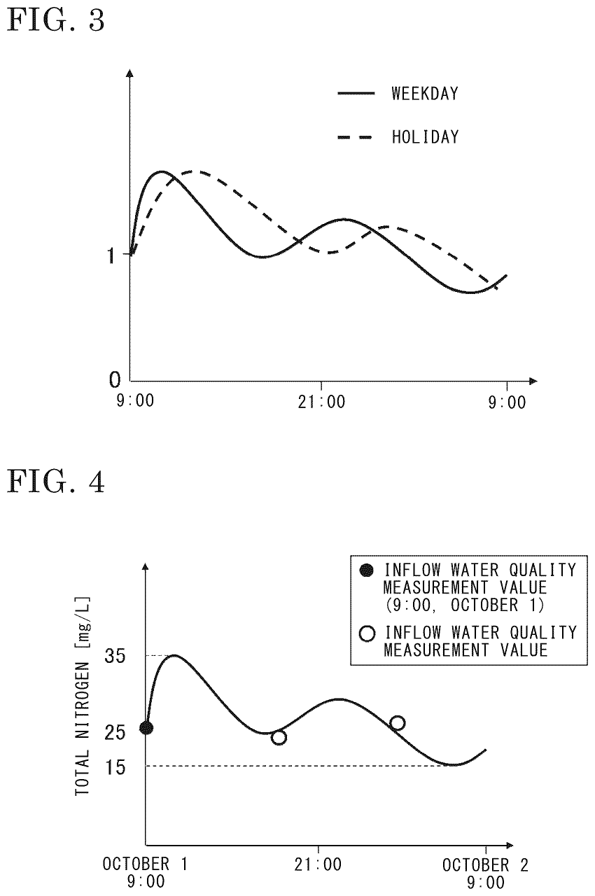

FIG. 3 illustrates an inflow water quality variation pattern acquired by the inflow water quality variation pattern acquisition unit 40. In a general sewage treatment plant, a certain pattern appears in variation in the inflow water quality through one day, The variation pattern differs among plant sites. For example, in a variation pattern characteristic to a residential district, peaks appear in the early morning and the evening, and in a variation pattern characteristic to an office district, a peak appears only during daytime. For example, through an all-day examination, inflow water on one day is sampled at intervals of one or two hours and the water quality thereof is measured, whereby variation in the inflow water quality value through the day is acquired a plurality of times in advance, and using an inflow water quality value at a given time, inflow water quality values at other times are normalized.

In FIG. 3, the vertical axis indicates the contaminant concentration in given treatment target water, and an example of temporal change, i.e., a variation pattern of the water quality, under normalization in which the inflow water quality value at 9:00 in the morning on a day is defined as 1. As described above, the variation pattern of the inflow water quality depends on an activity pattern of people in the treatment district, and thus, the variation pattern of the inflow water quality might differ between a weekday and a holiday, for example. Therefore, variation patterns of the inflow water quality are acquired separately for a weekday and a holiday in advance, and an inflow water quality variation pattern on the weekday indicated by a solid line and an inflow water quality variation pattern on the holiday indicated by a broken line in the graph are inputted to the inflow water quality variation pattern acquisition unit 40.

The above method in which the inflow water quality variation patterns are separated between a weekday and a holiday is merely an example. In accordance with the characteristic of the treatment district, inflow water quality variation patterns may be separated using a plurality of indices such as month, day, and the operation rates of factories in the treatment district. In addition, such an inflow water quality variation pattern does not necessarily need to be a one-day-basis pattern, and a pattern period such as one minute, one hour to one week, or one month may be selected in accordance with the variation cycle of the inflow water quality on each plant site. An inflow water quality variation pattern is inputted to the inflow water quality variation pattern acquisition unit 40 for every set pattern period. That is, in a case of an inflow water quality variation pattern on a one-day basis, an inflow water quality variation pattern is inputted to the inflow water quality variation pattern acquisition unit 40 on a one-day basis,

The instantaneous value of a contaminant concentration measured for inflow water sampled at a spot in the inflow section 15 or near the inflow section 15 in the bioreactor tank 10 is inputted to the inflow water quality measurement value acquisition unit 50 together with the sampling date and time. It is desirable that the kinds of contaminants for input to the inflow water quality measurement value acquisition unit 50 are the same as the kinds of contaminants for input to the inflow water quality variation pattern acquisition unit 40. Means for inputting the inflow water quality measurement value to the inflow water quality measurement value acquisition unit 50 may be any means, e. g., a tablet, mouse operation, keyboard operation, or input on a screen of a central monitoring system.

The frequency at which the inflow water quality measurement value is inputted to the inflow water quality measurement value acquisition unit 50 may be arbitrary, but in order to perform inflow water quality estimation with higher accuracy, it is desirable that the inflow water quality measurement value is inputted to the inflow water quality measurement value acquisition unit 50 in a cycle not longer than the cycle in which the inflow water quality variation pattern is inputted to the inflow water quality variation pattern acquisition unit 40. For example, in a case where the inflow water quality variation pattern is inputted to the inflow water quality variation pattern acquisition unit 40 on a one-day (24-hours) basis, it is desirable that a measurement value is inputted to the inflow water quality measurement value acquisition unit 50 at a frequency of one or more times a day.

The inflow water quality estimation unit 30 estimates an inflow water quality value subsequent to the sampling date and time of inflow water quality measurement, on the basis of the inflow water quality variation pattern transmitted from the inflow water quality variation pattern acquisition unit 40 via a signal line 40a and the measurement value of the inflow water quality transmitted from the inflow water quality measurement value acquisition unit 50 via a signal line 50a. The inflow water quality value estimated by the inflow water quality estimation unit 30 is transmitted to the target aeration amount calculation unit 60 via the signal line 30a. The target aeration amount calculation unit 60 calculates the target value for the aeration amount on the basis of the inflow water quality value estimated by the inflow water quality estimation unit 30.

Next, operation of the inflow water quality estimation unit 30 will be described. The inflow water quality estimation unit 30 includes a sampling date-and-time extraction unit 31, an inflow water quality variation pattern selection unit 32, and an inflow water quality calculation unit 33. The sampling date-and-time extraction unit 31 extracts a feature of the sampling date and time of the inflow water quality measurement value inputted to the inflow water quality measurement value acquisition unit 50, and associates the extracted feature with a feature of the date and time of the inflow water quality variation pattern stored in the inflow water quality variation pattern acquisition unit 40. For example, in a case where inflow water quality variation patterns for a weekday and a holiday have been inputted to the inflow water quality variation pattern acquisition unit 40 and an inflow water quality measurement value for a weekday is inputted to the inflow water quality measurement value acquisition unit 50, the sampling date-and-time extraction unit 31 extracts “weekday” as the feature of the sampling date and time.

The inflow water quality variation pattern selection unit 32 selects the variation pattern of the inflow water quality corresponding to the feature of the sampling date and time extracted by the sampling date-and-time extraction unit 31, from the inflow water quality variation patterns inputted to the inflow water quality variation pattern acquisition unit 40. For example, in a case where “weekday” is extracted as the feature of the sampling date and time as described above, the inflow water quality variation pattern for “weekday” is selected.

The inflow water quality calculation unit 33 calculates an inflow water quality value subsequent to the sampling date and time of inflow water quality measurement, on the basis of the variation pattern of the inflow water quality transmitted from the inflow water quality variation pattern selection unit 32 and the measurement value of the inflow water quality inputted to the inflow water quality measurement value acquisition unit 50. That is, with the sampling date and time of inflow water quality measurement defined as a first time, an inflow water quality value subsequent to the first time is calculated.

With reference to FIG. 4, an estimation value of the inflow water quality calculated by the inflow water quality calculation unit 33 will be described. For example, it is assumed that a variation pattern of an inflow total nitrogen concentration on a one-day basis is inputted to the inflow water quality variation pattern acquisition unit 40, and 25 mg/L is inputted as an inflow total nitrogen concentration at 9:00 on October 1, to the inflow water quality measurement value acquisition unit 50. In this case, the inflow water quality calculation unit 33 outputs the value of the inflow total nitrogen concentration for each time until 9:00 on October 2 which is one day later, starting from the measurement value of 25 mg/L at 9:00 on October 1. That is, a variation pattern indicated by a solid line in FIG. 4 is calculated.

For example, it is assumed that the sampling cycle is 8 hours and an inflow water quality measurement value is next inputted from the inflow water quality measurement value acquisition unit 50 at 17:00 on October 1. Then, since the day is still a weekday, the pattern is not changed in the inflow water quality variation pattern selection unit 32, but the value of the inflow total nitrogen concentration for each time until 9:00 on October 2 which is one day later is estimated again in accordance with the inputted inflow water quality measurement value. Similarly, also when an inflow water quality measurement value is inputted at 1:00 on October 2 which is further 8 hours later, estimation is performed again.

Here, an estimation algorithm implemented in the inflow water quality calculation unit 33 may be any algorithm that can estimate temporal variation in the inflow water quality, using the inflow water quality variation pattern and the inflow water quality measurement value as inputs, and for example, a linear/nonlinear regression model, machine learning, reinforcement learning, deep reinforcement learning, deep learning, random forest, a neural network, and another prediction method using artificial intelligence, may be used. The inflow water quality variation pattern obtained as a target of estimation can be used as a learned inflow water quality variation pattern to the inflow water quality variation pattern acquisition unit 40.

In a case where a variation pattern of the inflow total nitrogen concentration on a one-week basis is inputted to the inflow water quality variation pattern acquisition unit 40, inflow total nitrogen concentrations for respective times from 9:00 on October 1 to 9:00 on October 8 are outputted.

As described above, since variation patterns of the inflow water quality have been acquired in advance, it is not necessary to use a sensor capable of continuously measuring the inflow water quality. Further, an inflow water quality subsequent to the sampling date and time of inflow water quality measurement is estimated by combination with the inflow water quality measurement value, whereby accuracy of inflow water quality estimation can be improved in each sampling.

The target aeration amount calculation units 60 (61, 62, 63) calculate target values for the amounts of aeration to be supplied from the diffuser plates 11, 12, 13, on the basis of the inflow water quality value estimated by the inflow water quality estimation unit 30. The target aeration amount calculation units 61, 62, 63 calculate the target aeration amounts in association with the inflow water quality estimation value corresponding to the time at which the target aeration amounts are calculated. For example, calculation is performed on the basis of the following

### Formulae (1) to (3).

$$Qair 1 = K 1 \times TN \qquad ( 1 )$$

$$Qair 2 = K 2 \times TN \qquad ( 2 )$$

$$Qair 3 = K 3 \times TN \qquad ( 3 )$$

Here, Qair1, Qair2, and Qair3 are the target aeration amounts calculated by the target aeration amount calculation units 61, 62, 63, IN is the inflow water quality estimated by the inflow water quality estimation unit 30, and K1, K2, and K3 are proportionality constants.

By calculating the target aeration amounts on the basis of Formulae (1) to (3), it becomes possible to control the aeration amounts so as to follow variation in the inflow water quality. Thus, control delay is suppressed, whereby variation in the water quality after treatment is also suppressed and a favorable treated water quality is obtained, while an excessive aeration amount can be reduced. The values of K1, K2, and K3 do not necessarily need to be all equal to each other, and they may be set arbitrarily in accordance with the positions of the diffuser plates 11, 12, 13. In particular, in a case where the influence of variation in the inflow water quality is desired to be made as small as possible, the proportionality constants may be set so as to satisfy K1>K2>K3, whereby variation in the inflow water quality can be suppressed earlier on the preceding-stage side in the bioreactor tank 10. Formulae (1) to (3) are merely an example, and in a case of desiring to control the aeration amounts so as to follow an inflow load (inflow contaminant concentration % inflow water amount), a term of the inflow water amount may be added.

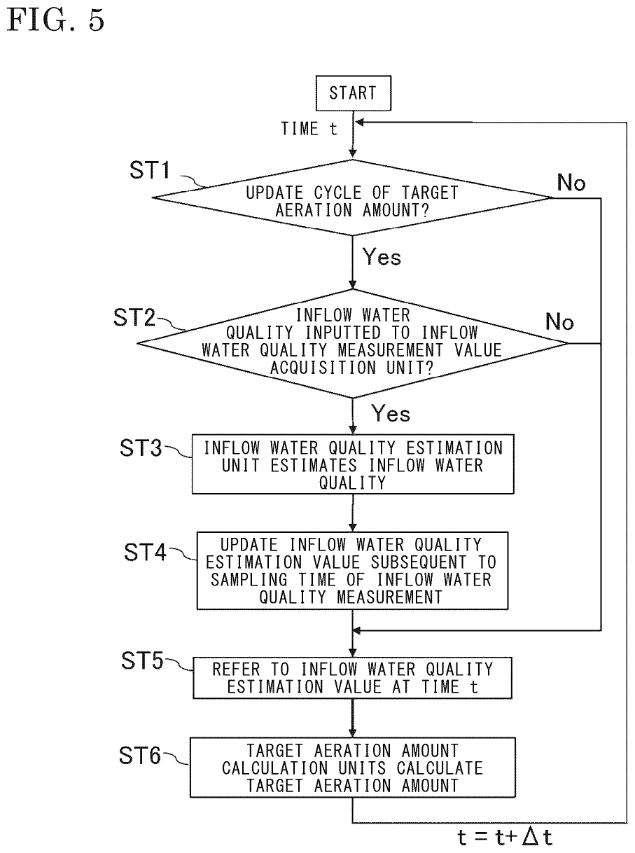

Next, an operation flow of the aeration amount control device in embodiment 1 will be described with reference to FIG. 5.

Variation patterns of the inflow water quality have been inputted to the inflow water quality variation pattern acquisition unit 40 in advance. From this state, control by the aeration amount control device is started.

The operation flow of the aeration amount control device includes six steps ST1 to ST6 shown in FIG. 5. After the aeration amount control is started, the flow through steps ST1 to ST6 is repeated at certain time intervals At. Here, Δt is set at a value not greater than an update cycle T of the target aeration amount of the target aeration amount calculation unit 60, and it is desirable that Δt is about one second to one minute. However, a period longer than a period required for going through all the steps ST1 to ST6 needs to be set as At.

First, in step ST1, whether or not time t corresponds to the update cycle of the target aeration amount of the target aeration amount calculation unit 60, is determined. In a case where the update cycle T of the target aeration amount is 5 minutes, step ST1 gives determination as Yes at 5-minute intervals. In a case of Yes in step ST1, the process proceeds to the next step ST2, and in a case of No in step ST1, the process proceeds to step ST5.

In step ST2, whether or not a measurement value of an inflow water quality has been newly inputted to the inflow water quality measurement value acquisition unit 50 during a period to time t from time t−Δt which is a time preceding by one cycle of the repetition interval Δt, is determined. In a case where the input cycle of the inflow water quality to the inflow water quality measurement value acquisition unit 50 is one day, step ST2 gives determination as Yes at one-day intervals. In a case of Yes in step ST2, the process proceeds to the next step ST3, and in a case of No in step ST2, the process proceeds to step ST5.

Here, after water for inflow water quality measurement is sampled, water quality measurement is performed, and a result thereof is inputted to the inflow water quality measurement value acquisition unit 50. Therefore, the time at which the sampling for inflow water quality measurement was performed is before the time t. For example, it is assumed that the sampling for inflow water quality measurement is performed at 9:00 on October 1, and then, after one hour is taken for water quality analysis, the measurement value at 9:00 on October 1 is inputted to the inflow water quality measurement value acquisition unit 50. In this case, the inflow water quality measurement value at the time of 9:00 on October 1 is inputted when the time t in the operation flow of the aeration amount control device is 10:00 on October 1.

Next, in step ST3, the inflow water quality estimation unit 30 estimates an inflow water quality value subsequent to the sampling date and time of inflow water quality measurement. In the present embodiment, as shown in FIG. 4, the inflow water quality variation pattern on a one-day basis has been input to the inflow water quality variation pattern acquisition unit 40. Therefore, variation in the inflow water quality value from 9:00 on October 1 to 9:00 on October 2 is estimated,

Next, in step ST4, the inflow water quality estimation value recorded at time t−Δt is updated to the inflow water quality estimation value estimated in step ST3.

In step ST5, the target aeration amount calculation units 61, 62, 63 refer to the inflow water quality estimation value at the time t. In the present embodiment, in a case where the time t is 10:00 on October 1, the inflow water quality estimation value at 10:00 on October 1 is referred to.

In step ST6, using the inflow water quality estimation value referred to in step ST5 and Formulae (1) to (3), each target aeration amount calculation unit 61, 62, 63 calculates a target aeration amount at time t. The calculated target aeration amounts are transmitted to the air volume adjustment valves 71, 72, 73 via the signal lines 61a, 62a, 63a, and the opening degrees of the air volume adjustment valves 71, 72, 73 are adjusted so as to achieve the target aeration amounts.

In the operation flow of the aeration amount control device shown in FIG. 5, the water quality of the treatment target water flowing into the bioreactor tank 10 at 9:00 on October 1 which is the first time is sampled, to perform water quality measurement. Thereafter, at 10:00 on October 1 which is a second time, the inflow water quality measurement value acquisition unit 50 acquires an inflow water quality measurement value at the first time, and the inflow water quality estimation unit 30 estimates an inflow water quality subsequent to the sampling date and time of inflow water quality measurement. Here, since the acquisition cycle for the water quality measurement value by the inflow water quality measurement value acquisition unit 50 is one day, the water quality of the treatment target water flowing into the bioreactor tank 10 at 9:00 on October 2 which is a third time is sampled, to perform water quality measurement. Thereafter, at 10:00 on October 2 which is a fourth time, the inflow water quality measurement value acquisition unit 50 acquires an inflow water quality measurement value at the third time, and the inflow water quality estimation unit 30 estimates an inflow water quality subsequent to the sampling date and time (third time) of inflow water quality measurement. In this way, sampling of the inflow water quality, acquisition of the measurement value, and estimation of the inflow water quality are repeated. The third time may be before 9:00 on October 2, as long as sampling and determination of the measurement value are performed so that an inflow water quality measurement value subsequent to the first time can be acquired at the fourth time. As a matter of course, a newer inflow water quality measurement value can be acquired if the third time is closer to 9:00 on October 2,

As described above, in the water treatment system according to the present embodiment 1, the inflow water quality estimation unit 30 estimates an inflow water quality value on the basis of the inflow water quality variation pattern inputted to the inflow water quality variation pattern acquisition unit 40 and the inflow water quality measurement value inputted to the inflow water quality measurement value acquisition unit 50, whereby it becomes possible to estimate the inflow water quality value with high accuracy while reflecting both of the most recent water quality measurement value and difference in the inflow water quality variation pattern depending on the sampling date and time. Further, the target aeration amount calculation units 61, 62, 63 calculate the target aeration amounts, using the estimated inflow water quality value, and adjust the air volume adjustment valves 71, 72, 73 in accordance with the calculated target aeration amounts. Thus, it is possible to perform treatment according to the water quality value of inflow water while suppressing control delay, whereby it becomes possible to reduce an excessive aeration amount while obtaining a favorable treated water quality.

Conventionally, an operator who measures the water quality and an operator who performs operation control for the water treatment system are different, and thus it takes time to reflect a water quality result in control for the water treatment system. However, as in the present embodiment, control for the water treatment system is performed using the estimated inflow water quality estimation value, whereby it becomes possible to perform control in accordance with the water quality.

### Embodiment 2

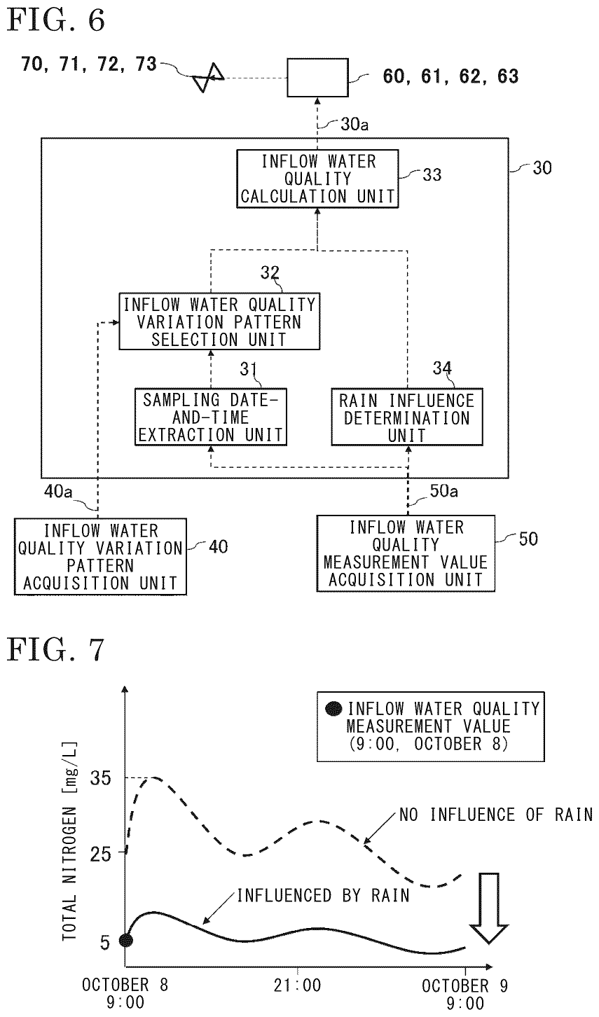

Hereinafter, a water treatment system according to embodiment 2 will be described with reference to FIG. 6.

FIG. 6 is a block diagram showing the configuration of an aeration amount control device of the water treatment system according to embodiment 2. Difference from embodiment 1 is that the inflow water quality estimation unit 30 includes a rain influence determination unit 34. The other configurations are the same as those in embodiment 1 and therefore the description thereof is omitted.

In FIG. 6, the rain influence determination unit 34 determines whether or not the inflow water quality measurement value inputted to the inflow water quality measurement value acquisition unit 50 has been influenced by rain, and transmits the inflow water quality measurement value inputted to the inflow water quality measurement value acquisition unit 50 and a rain influence degree to the inflow water quality calculation unit 33.

In rainy weather, the treatment target water is diluted by rainwater, so that the contaminant concentration in the inflow water becomes smaller than in fine weather, and thus estimation accuracy for the inflow water quality might be deteriorated. Therefore, in a case where the inflow water quality measurement value is smaller than a determination threshold preset in the rain influence determination unit 34, the rain influence determination unit 34 calculates a rain influence degree. In calculation of the rain influence degree, weather information from a weather radar in a target treatment district of the plant site may be acquired to determine the influence of rain, and a result thereof may be used.

The rain influence degree is a ratio of dilution of inflow water due to rain entry water, and is calculated by Formula (4).

$$Dilution ratio = 1 - Ssp\_r / Ssp\_ave \qquad ( 4 )$$

Here, Ssp_r is an inflow water quality measurement value sampled at the date and time when there is an influence of rain, and Ssp_ave is an average value of inflow water quality measurement values in fine weather. As the average value of the inflow water quality measurement values, an inflow water quality measurement value at the same sampling date and time is acquired a plurality of times, and the average value thereof is inputted to the rain influence determination unit 34.

Next, an estimation value of an inflow water quality calculated by the inflow water quality calculation unit 33 in a case where the rain influence determination unit 34 determines that there is an influence of rain, will be described with reference to FIG. 7.

For example, it is assumed that a variation pattern of an inflow total nitrogen concentration on a one-day basis is inputted to the inflow water quality variation pattern acquisition unit 40, 5 mg/L is inputted as an inflow total nitrogen concentration at 9:00 on October 8, to the inflow water quality measurement value acquisition unit 50, and the rain influence determination unit 34 determines that “there is an influence of rain”. In this case, the inflow water quality calculation unit 33 outputs the value of the inflow total nitrogen concentration for each time until 9:00 on October 9 which is one day later, starting from the inflow water quality measurement value of 5 mg/L at 9:00 on October 8. Here, in accordance with the dilution ratio due to rain, estimation is performed so that the estimation value of the inflow total nitrogen concentration becomes smaller and the variation width of the inflow water quality value also becomes smaller, than in a case where there is no influence of rain.

A broken line in FIG. 7 indicates a variation pattern of an estimated inflow total nitrogen concentration on a weekday in a case where the weather is fine, i.e., there is no influence of rain. A future variation is estimated from the dilution ratio and the inflow water quality measurement value transmitted from the rain influence determination unit 34, whereby a pattern indicated by a solid line can be obtained. In this way, it is possible to estimate the inflow water quality value accurately even on a day when there is an influence of rain. An estimation algorithm implemented in the inflow water quality calculation unit 33 may be any algorithm that can estimate temporal variation in the inflow water quality value, using the inflow water quality variation pattern, the inflow water quality measurement value, and the dilution ratio due to rain, as inputs, and for example, a linear/nonlinear regression model, machine learning, reinforcement learning, deep reinforcement learning, deep learning, random forest, a neural network, and another prediction method using artificial intelligence, may be used as in embodiment 1.

The target aeration amount calculation units 61, 62, 63 calculate target values for the amounts of aeration to be supplied from the diffuser plates 11, 12, 13, on the basis of the inflow water quality value estimated by the inflow water quality estimation unit 30. The target aeration amount calculation units 61, 62, 63 calculate the target aeration amounts in association with the inflow water quality estimation value corresponding to the time at which the target aeration amounts are calculated. In general, in a case where there is an influence of rain, the contaminant concentration in inflow water is extremely small, and therefore, if the same aeration amounts as in fine weather are used, excessive aeration would be supplied. In the present embodiment, the target aeration amount calculation units 61, 62, 63 calculate the target aeration amounts on the basis of the estimation value of the inflow water quality for which the influence of rain has been considered. Thus, it is possible to not only suppress variation in the inflow water quality and stabilize the treated water quality but also further reduce excessive aeration as compared to a case of fine weather.

As described above, according to embodiment 2, the inflow water quality estimation unit 30 estimates the inflow water quality, using the rain influence degree determined by the rain influence determination unit 34. Thus, it becomes possible to estimate high-accuracy inflow water quality value even in a case where an inflow water quality variation pattern different from a normal one in fine weather is predicted from a measured inflow water quality value, Further, the target aeration amount calculation units 61, 62, 63 calculate target aeration amounts, using the inflow water quality estimation value for which the influence of rain has been considered, and adjust the air volume adjustment valves 71, 72, 73 in accordance with the calculated target aeration amounts. Thus, it is possible to perform treatment according to the water quality of inflow water even in rainy weather, whereby it becomes possible to reduce an excessive aeration amount while obtaining a favorable treated water quality.

### Embodiment 3

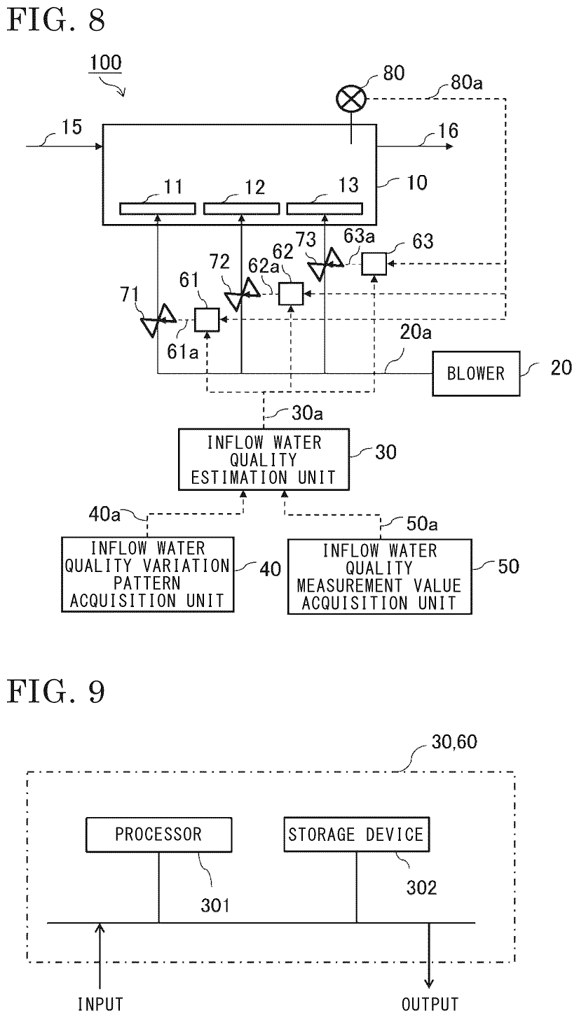

Hereinafter, a water treatment system according to embodiment 3 will be described with reference to FIG. 8.

FIG. 8 is a block diagram showing the configuration of the water treatment system according to embodiment 3. Difference from embodiment 1 is that a contaminant concentration measurement unit 80 having a concentration measurement instrument is provided for measuring a contaminant concentration at a time point when treatment in the bioreactor tank 10 for the treatment target water flowing into the bioreactor tank 10 is finished. The other configurations are the same as those in embodiment 1 or 2 and the description thereof is omitted.

The contaminant concentration measurement unit 80 according to the present embodiment 3 corresponds to a second water quality measurement value acquisition unit different from the inflow water quality measurement value acquisition unit 50. A main role of the contaminant concentration measurement unit 80 is to measure a contaminant concentration at a time point when treatment is finished, and therefore it is desirable that the concentration measurement instrument thereof is provided at a position closer to the outflow section 16 in the bioreactor tank 10. Alternatively, the concentration measurement instrument of the contaminant concentration measurement unit 80 may be provided at the outflow section 16.

For the concentration measurement instrument, one or more kinds are selected from treatment targets in the bioreactor tank 10. Examples include BOD, COD, ammonia nitrogen, total nitrogen, Kjeldahl nitrogen, total phosphorus, and phosphate phosphorus, The contaminant concentration measurement unit 80 does not necessarily need to have a concentration measurement instrument for the same kind as the water quality inputted to the inflow water quality variation pattern acquisition unit 40 and the inflow water quality measurement value acquisition unit 50, and may have a concentration measurement instrument for a desired kind of treated water quality to be monitored.

The contaminant concentration measured by the contaminant concentration measurement unit 80 is transmitted to the target aeration amount calculation units 61, 62, 63 via a signal line 80a. The target aeration amount calculation units 61, 62, 63 calculate target values for aeration amounts on the basis of the estimation value of the inflow water quality estimated by the inflow water quality estimation unit 30 and the contaminant concentration measured by the contaminant concentration measurement unit 80,

Next, a calculation method for target values for aeration amounts by the target aeration amount calculation units 61, 62, 63 will be described. Hereinafter, it is assumed that a variation pattern of an inflow water total nitrogen concentration and an inflow water quality measurement value are respectively inputted to the inflow water quality variation pattern acquisition unit 40 and the inflow water quality measurement value acquisition unit 50, and the contaminant concentration measurement unit 80 has an ammonia nitrogen concentration meter as a contaminant concentration meter.

The target values for the aeration amounts are determined through control for following variation in the inflow water quality value estimated by the inflow water quality estimation unit 30 and control (proportional integral (PI) control) for performing operation so that the contaminant concentration measured by the contaminant concentration measurement unit 80 becomes equal to a certain target water quality, As a specific example, the target values of the aeration amounts are determined on the basis of Formulae (5) to (7) shown below. Among components of a control unit in the present embodiment 3, a target water quality setting unit for setting a target water quality is not shown, but the target aeration amount calculation units 61, 62, 63 have a target water quality setting unit therein, and an operator can set a target water quality from the outside.

$$Qair 1 = K 1 \times TN + Kp 1 \times ( NH 4 - NH {4}^{*} ) + Ki 1 \times \sum ( NH 4 - NH {4}^{*} ) \qquad ( 5 )$$

$$Qair 2 = K 2 \times TN + Kp 2 \times ( NH 4 - NH {4}^{*} ) + Ki 2 \times \sum ( NH 4 - NH {4}^{*} ) \qquad ( 6 )$$

$$Qair 3 = K 3 \times TN + Kp 3 \times ( NH 4 - NH {4}^{*} ) + Ki 3 \times \sum ( NH 4 - NH {4}^{*} ) \qquad ( 7 )$$

Here, Qair1, Qair2, and Qair3 are target aeration amounts calculated by the target aeration amount calculation units 61, 62, 63, TN is the estimation value of the inflow water total nitrogen concentration estimated by the inflow water quality estimation unit 30, K1, K2, and K3 are proportionality constants, Kp1, Kp2, and Kp3 are proportional gains (constants), Ki1, Ki2, and Ki3 are integral gains (constants), NH4 is an ammonia nitrogen concentration measured by the contaminant concentration measurement unit 80, and NH4* is a target value for the ammonia nitrogen concentration. In addition, E represents the sum of measurement values of (NH4-NH4*) after calculation of aeration amounts by Formulae (5) to (7) is started. For example, in a case where calculation of aeration amounts based on Formulae (5) to (7) is performed at one-minute intervals, the value of X (NH4-NH4*) after one hour is the sum of 60 measurement values of (NH4-NH4*) obtained per one minute from a time just after calculation of aeration amounts by Formulae (5) to (7) is started.

The first terms in Formulae (5) to (7) represent proportional control for the estimation value of the inflow water total nitrogen concentration estimated by the inflow water quality estimation unit 30, whereby it becomes possible to control the aeration amounts so as to follow variation in the inflow water quality.

The second terms and the third terms in Formulae (5) to (7) represent PI control based on a difference between the ammonia nitrogen concentration measured by the contaminant concentration measurement unit 80 and the target value for ammonia nitrogen, whereby the aeration amounts are controlled so that the treated water quality becomes constant with respect to the target value, and thus necessary amounts of aeration can be supplied to the bioreactor tank 10 without excess or deficiency. The proportionality constants (K1, K2, K3), the proportional gains (Kp1, Kp2, Kp3), and the integral gains (Ki1, Ki2, Ki3) for the estimation value of the inflow water quality need not be all equal among the target aeration amount calculation units 61, 62, 63, and may be set at arbitrary values in accordance with the positions of the diffuser plates 11, 12, 13. In particular, in a case of desiring to make the influence of variation in the inflow water quality value as small as possible, the proportionality constants are set so as to satisfy K1>K2>K3, whereby variation in the inflow water quality value can be suppressed early on the preceding-stage side in the bioreactor tank 10. In addition, in a case of desiring to make

variation in the treated water quality value as small as possible, setting is made so as to satisfy Kp1<Kp2<Kp3 and Ki1<Ki2<Ki3, whereby the influence of PI control can be made greater for the air volume adjustment valve that is closer to the location where the contaminant concentration measurement unit 80 is provided. Formulae (5) to (7) are merely an example, and in a case of desiring to control the aeration amounts so as to follow an inflow load (inflow contaminant concentration x inflow water amount), a term of an inflow water amount may be added at the first term.

As described above, according to embodiment 3, the target aeration amount calculation units 61, 62, 63 calculate target aeration amounts, using the inflow water quality estimation value estimated by the inflow water quality estimation unit 30 and the contaminant concentration measured by the contaminant concentration measurement unit 80, whereby the treated water quality can be controlled to be constant with improved response of the aeration amounts with respect to variation in the inflow water quality, without using a sensor capable of continuously measuring the inflow water quality. Thus, it is possible to reduce an excessive aeration amount by supplying aeration without excess or deficiency, while suppressing variation in the treated water quality value and obtaining a favorable treated water quality.

In the above embodiments 1 to 3, the inflow water quality estimation unit 30 and the target aeration amount calculation unit 60 of the water treatment system 100 are composed of a processor 301 and a storage device 302, as shown in a hardware example in FIG. 9. Although not shown, the storage device includes a volatile storage device such as a random access memory, and a nonvolatile auxiliary storage device such as a flash memory. The storage device may include an auxiliary storage device of a hard disk, instead of a flash memory. The processor 301 executes a program inputted from the storage device 302. In this case, the program is inputted from the auxiliary storage device to the processor 301 via the volatile storage device. The processor 301 may output data such as a calculation result to the volatile storage device of the storage device 302, or may store such data into the auxiliary storage device via the volatile storage device.

### Embodiment 4

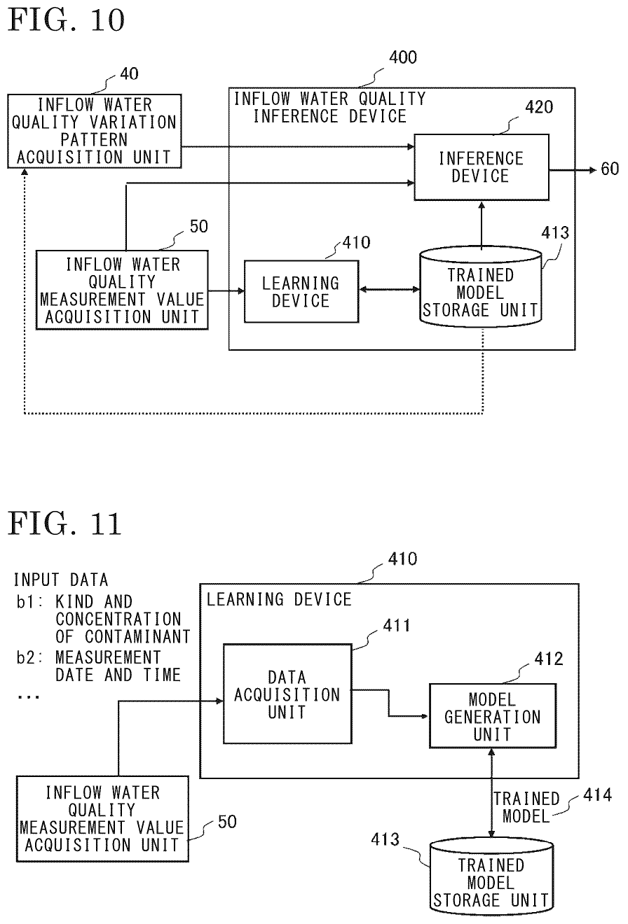

Hereinafter, a water treatment system according to embodiment 4 will be described with reference to FIG. 10 to FIG. 14. In the present embodiment, an inflow water quality inference device in which the inflow water quality estimation unit 30 shown in each of embodiments 1 to 3 is implemented with a machine learning device provided, will be described.

FIG. 10 shows the configuration of an inflow water quality inference device 400 of the water treatment system according to the present embodiment 4, and the inflow water quality inference device 400 includes a learning device 410 and an inference device 420. The inflow water quality inference device 400 may be the inflow water quality estimation unit 30. Hereinafter, a procedure for inferring an inflow water quality value will be described, with the procedure separated into a “learning phase” and a “utilization phase” for actually performing inference.

### <Learning Phase>

FIG. 11 shows the configuration of a learning device 410. The learning device 410 includes a data acquisition unit 411, a model generation unit 412, and a trained model storage unit 413.

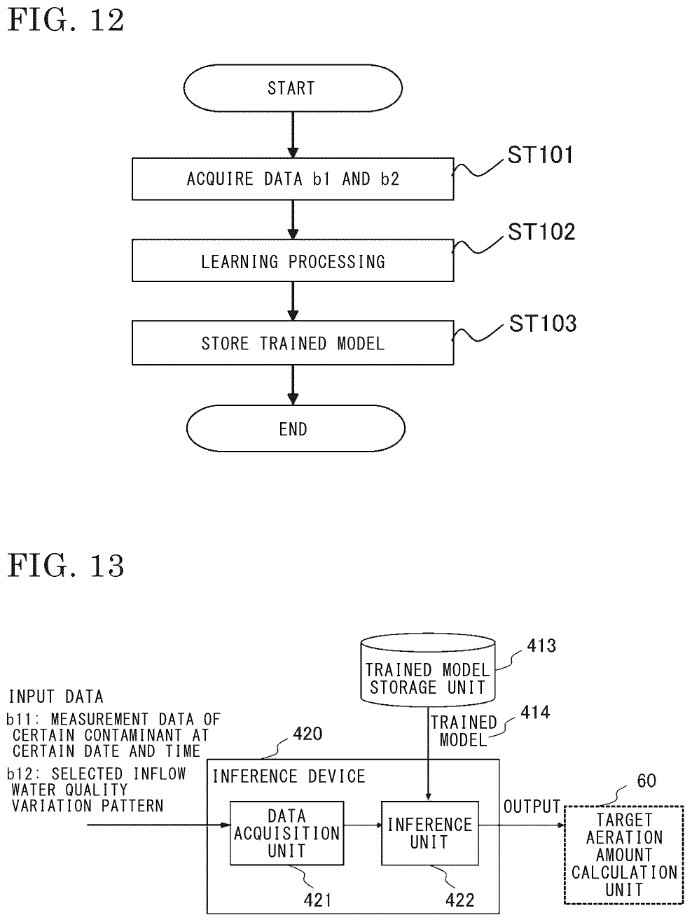

FIG. 12 is a flowchart showing a processing procedure for executing the learning phase using the learning device 410.

The data acquisition unit 411 acquires data b1 of the kind and the concentration of a contaminant and data b2 of the measurement date and time, as input data, from the inflow water quality measurement value acquisition unit 50, and combines both data to obtain time-series training data (step ST101).

The model generation unit 412 learns variation in the inflow water quality on the basis of the training data outputted from the data acquisition unit 411 (step ST102). That is, from a plurality of time-series data of the concentrations of contaminants over a certain period, for example, water quality variation for each kind of contaminants on each day (weekday or holiday) is learned to generate a trained model 414 including water quality variation patterns. In learning for generating the trained model 414, for example, a linear/nonlinear regression model, machine learning, reinforcement learning, deep reinforcement learning, deep learning, random forest, a neural network, and another prediction method using artificial intelligence, may be used.

By executing learning as described above, the model generation unit 412 generates and outputs the trained model 414, and the trained model storage unit 413 stores the trained model 414 outputted from the model generation unit 412 (step ST103).

### <Utilization Phase>

FIG. 13 shows the configuration of the inference device 420. The inference device 420 includes a data acquisition unit 421 and an inference unit 422.

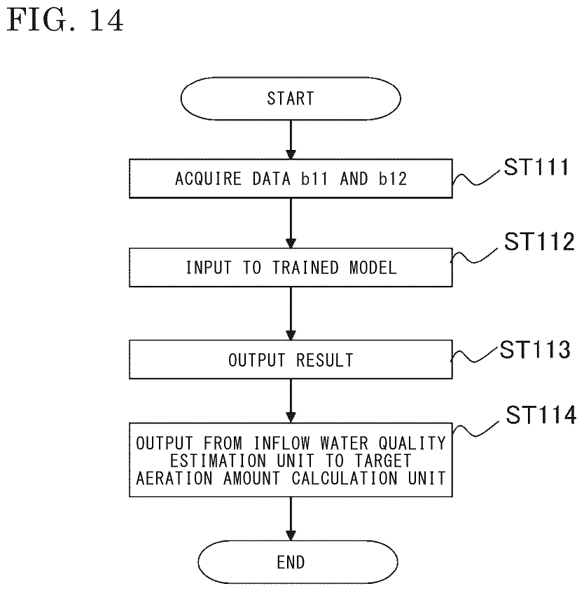

FIG. 14 is a flowchart showing a processing procedure for executing the utilization phase for inferring a water quality value of inflow water using the inference device 420.

The data acquisition unit 421 acquires water quality measurement data b11 of a contaminant from the inflow water quality measurement value acquisition unit 50 and an inflow water quality variation pattern b12 selected by the inflow water quality variation pattern selection unit 32, as input data (step ST111).

The inference unit 422 infers variation in the inflow water quality, using the trained model 414. That is, new water quality measurement data b11 of a contaminant at a given time point acquired by the data acquisition unit and a selected inflow water quality variation pattern b12, are inputted to the trained model 414 (step ST112), whereby subsequent water quality variation values can be inferred with high accuracy and can be outputted from the inference device 420 (step ST113).

A result of inference of inflow water quality values is outputted to the target aeration amount calculation unit 60 (step ST114).

The water quality measurement data b11 of a contaminant from the inflow water quality measurement value acquisition unit 50 and the inflow water quality variation pattern b12 selected by the inflow water quality variation pattern selection unit 32 are inputted from the data acquisition unit 421 to the inference unit 422. However, the inflow water quality variation pattern b12 selected by the inflow water quality variation pattern selection unit 32 merely serves as reference data, and may be omitted. This is because the water quality measurement data b11 of a contaminant acquired from the inflow water quality measurement value acquisition unit 50 is imparted with water quality information such as the date and time of measurement and the kind of the contaminant and thus can be applied to the trained data.

In the above description, the example corresponding to embodiment 1 has been described. However, as a matter of course, the inflow water quality inference device 400 for rainy weather as in embodiment 2 can be constructed by including rain information in data to be inputted to the inflow water quality inference device 400.

In addition, as a matter of course, the inflow water quality inference device 400 is also applicable to embodiment 3.

The inference device 420 may be the inflow water quality estimation unit 30, and the learning device 410 and the trained model storage unit 413 may be externally provided to the inflow water quality estimation unit 30 of each of embodiments 1 to 3, or these may be integrated as the inflow water quality inference device 400 as shown in FIG. 10.

An example of hardware of the inflow water quality inference device 400 is composed of a processor and a storage device as in the configuration shown in FIG. 9, and therefore the description thereof is omitted. The processor executes a program to implement the functions of the learning device 410 and the inference device 420 of the inflow water quality inference device 400.

As the patterns to be inputted to the inflow water quality variation pattern acquisition unit 40 in each of embodiments 1 to 4, inflow water quality variation patterns based on the trained model may be used.

In the present embodiment 4, it has been described that an inference result of the inflow water quality is outputted using the trained model 414 trained in the model generation unit 412. However, a trained model may be acquired from the outside and an inference result of the inflow water quality may be outputted on the basis of the acquired trained model.

In the present embodiment 4, regarding the learning phase and the utilization phase described separately from each other, the learning phase may be carried out first, and then the utilization phase may be carried out, or both phases may be carried out in parallel. In a case of carrying out both phases in parallel, a threshold for completion of learning, e.g., the number of acquired data, may be set. Then, only learning may be carried out during a period in which learning has not been completed yet, and after learning is completed, both phases may be carried out in parallel.

As described above, according to embodiment 4, effects of embodiment 1 are provided, and in addition, data b1 of the kind and the concentration of a contaminant and data b2 of the measurement date and time are acquired from the inflow water quality measurement value acquisition unit 50, and both data are combined to obtain time-series training data, thus generating a trained model, and new present water quality measurement data b11 of a contaminant at a given time point and a selected inflow water quality variation pattern b12 are inputted to the trained model, whereby subsequent water quality variation values can be inferred with high accuracy. Thus, it becomes possible to perform aeration amount control with higher accuracy on the basis of a result of the above inference.

Although the disclosure is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations to one or more of the embodiments of the disclosure.

It is therefore understood that numerous modifications which have not been exemplified can be devised without departing from the scope of the present disclosure. For example, at least one of the constituent components may be modified, added, or eliminated. At least one of the constituent components mentioned in at least one of the preferred embodiments may be selected and combined with the constituent components mentioned in another preferred embodiment.

### DESCRIPTION OF THE REFERENCE CHARACTERS

10 bioreactor tank
11, 12, 13 diffuser plate
15 inflow section
16 outflow section
20 blower
20a pipe
30 inflow water quality estimation unit
30a signal line
31 sampling date-and-time extraction unit
32 inflow water quality variation pattern selection unit
33 inflow water quality calculation unit
34 rain influence determination unit
40 inflow water quality variation pattern acquisition unit
40a signal line
50 inflow water quality measurement value acquisition unit
50a signal line
60, 61, 62, 63 target aeration amount calculation unit
61a, 62a, 63a signal line
70, 71, 72, 73 air volume adjustment valve
80 contaminant concentration measurement unit
80a signal line
100 water treatment system
301 processor
302 storage device
400 inflow water quality inference device
410 learning device
411 data acquisition unit
412 model generation unit
413 trained model storage unit
414 trained model
420 inference device
421 data acquisition unit
422 inference unit

1. A water treatment system for performing water treatment through biological oxidation while performing aeration from a blower to a reaction tank, the water treatment system comprising:
a first water quality measurement value acquisition circuitry which acquires a water quality measurement value at a first time point, of treatment target water flowing into the reaction tank;
a water quality variation pattern acquisition circuitry which, from time-series change in water quality information of the treatment target water acquired in advance, acquires a plurality of water quality variation patterns each according to a condition at a time of acquisition of the water quality information acquired in advance;
an inflow water quality estimation circuitry which selects one water quality variation pattern that matches a condition at a time of acquisition of the water quality measurement value at the first time point, from the plurality of water quality variation patterns included in the water quality variation pattern acquisition circuitry, and estimates an inflow water quality value subsequent to the first time point on the basis of the selected one water quality variation pattern and the water quality measurement value at the first time point acquired by the first water quality measurement value acquisition circuitry; and
a control circuitry which controls an aeration amount of the blower subsequent to the first time point, on the basis of the inflow water quality value estimated by the inflow water quality estimation circuitry.

2. The water treatment system according to claim 1, further comprising a second water quality measurement value acquisition circuitry, wherein
the second water quality measurement value acquisition circuitry acquires a water quality measurement value of treated water that has been treated in the reaction tank, and
the control circuitry further controls the aeration amount of the blower subsequent to the first time point, on the basis of the water quality measurement value of the treated water acquired by the second water quality measurement value acquisition circuitry.

3. The water treatment system according to claim 1, wherein
the inflow water quality estimation circuitry includes
a learning device including a data acquisition circuitry which acquires time-series change in water quality information of the treatment target water, as training data, in advance, and a model generation circuitry which generates a trained model for inferring a plurality of the water quality variation patterns each of which is time-series change according to a condition at a time of acquisition of the water quality information, using the training data, and
an inference device which infers the inflow water quality value subsequent to the first time point, from the water quality measurement value at the first time point of the treatment target water acquired by the first water quality measurement value acquisition circuitry, using the trained model.

4. An aeration amount control device used for a water treatment system for performing water treatment through biological oxidation while performing aeration from a blower to a reaction tank, the aeration amount control device comprising:
a first water quality measurement value acquisition circuitry which acquires a water quality measurement value at a first time point, of treatment target water flowing into the reaction tank;
a water quality variation pattern acquisition circuitry which, from time-series change in water quality information of the treatment target water acquired in advance, acquires a plurality of water quality variation patterns each according to a condition at a time of acquisition of the water quality information acquired in advance;
an inflow water quality estimation circuitry which selects one water quality variation pattern that matches a condition at a time of acquisition of the water quality measurement value at the first time point, from the plurality of water quality variation patterns included in the water quality variation pattern acquisition circuitry, and estimates an inflow water quality value subsequent to the first time point on the basis of the selected one water quality variation pattern and the water quality measurement value at the first time point acquired by the first water quality measurement value acquisition circuitry; and
a target aeration amount calculation circuitry which calculates a target aeration amount of aeration to be supplied from the blower to the reaction tank subsequent to the first time point, on the basis of the inflow water quality value estimated by the inflow water quality estimation circuitry.

5. The aeration amount control device according to claim 4, wherein
the first water quality measurement value acquisition circuitry acquires a water quality measurement value of treatment target water flowing into the reaction tank at a second time point subsequent to the first time point, and
the inflow water quality estimation circuitry selects the water quality variation pattern from the water quality variation pattern acquisition circuitry, correspondingly to acquisition date and time of the water quality measurement value at the second time point acquired by the first water quality measurement value acquisition circuitry, and estimates an inflow water quality value subsequent to the second time point.

6. The aeration amount control device according to claim 4, wherein
the inflow water quality estimation unit includes a rain influence determination circuitry which determines whether the water quality measurement value acquired by the first water quality measurement value acquisition circuitry has been influenced by rain, and
in a case where the rain influence determination circuitry determines that the water quality measurement value has been influenced, the inflow water quality value subsequent to the first time point is estimated on the basis of a rain influence degree and the water quality variation pattern selected from the water quality variation pattern acquisition circuitry.

7. The aeration amount control device according to claim 4, further comprising a second water quality measurement value acquisition circuitry, wherein
the second water quality measurement value acquisition circuitry acquires a water quality measurement value of treated water that has been treated in the reaction tank, and
the target aeration amount calculation circuitry calculates the target aeration amount of aeration to be supplied from the blower to the reaction tank, on the basis of the inflow water quality value estimated by the inflow water quality estimation circuitry and the water quality measurement value of the treated water acquired by the second water quality measurement value acquisition circuitry.

8. An aeration amount control method used for a water treatment system for performing water treatment through biological oxidation while performing aeration from a blower to a reaction tank, the aeration amount control method comprising:
a water quality variation pattern acquisition step of, from time-series change in water quality information of treatment target water flowing into the reaction tank in advance, acquiring a plurality of water quality variation patterns according to a condition at a time of acquisition of the water quality information acquired in advance;
a water quality measurement value acquisition step of acquiring a water quality measurement value at a first time point, of the treatment target water flowing into the reaction tank;
an inflow water quality estimation step of selecting one water quality variation pattern that matches an acquisition condition for the water quality measurement value at the first time point, from a plurality of the water quality variation patterns acquired in the water quality variation pattern acquisition step, and estimating an inflow water quality value subsequent to the first time point on the basis of the selected one water quality variation pattern and the water quality measurement value at the first time point acquired in the first water quality measurement value acquisition step; and
a target aeration amount calculation step of calculating a target aeration amount of aeration to be supplied from the blower to the reaction tank subsequent to the first time point, on the basis of the inflow water quality value estimated in the inflow water quality estimation step.

9. The water treatment system according to claim 2, wherein
the inflow water quality estimation circuitry includes
a learning device including a data acquisition circuitry which acquires time-series change in water quality information of the treatment target water, as training data, in advance, and a model generation circuitry which generates a trained model for inferring a plurality of the water quality variation patterns each of which is time-series change according to a condition at a time of acquisition of the water quality information, using the training data, and
an inference device which infers the inflow water quality value subsequent to the first time point, from the water quality measurement value at the first time point of the treatment target water acquired by the first water quality measurement value acquisition circuitry, using the trained model.

10. The aeration amount control device according to claim 5, wherein
the inflow water quality estimation unit includes a rain influence determination circuitry which determines whether the water quality measurement value acquired by the first water quality measurement value acquisition circuitry has been influenced by rain, and
in a case where the rain influence determination circuitry determines that the water quality measurement value has been influenced, the inflow water quality value subsequent to the first time point is estimated on the basis of a rain influence degree and the water quality variation pattern selected from the water quality variation pattern acquisition circuitry.

11. The aeration amount control device according to claim 5, further comprising a second water quality measurement value acquisition circuitry, wherein
the second water quality measurement value acquisition circuitry acquires a water quality measurement value of treated water that has been treated in the reaction tank, and
the target aeration amount calculation circuitry calculates the target aeration amount of aeration to be supplied from the blower to the reaction tank, on the basis of the inflow water quality value estimated by the inflow water quality estimation circuitry and the water quality measurement value of the treated water acquired by the second water quality measurement value acquisition circuitry.

12. The aeration amount control device according to claim 6, further comprising a second water quality measurement value acquisition circuitry, wherein
the second water quality measurement value acquisition circuitry acquires a water quality measurement value of treated water that has been treated in the reaction tank, and
the target aeration amount calculation circuitry calculates the target aeration amount of aeration to be supplied from the blower to the reaction tank, on the basis of the inflow water quality value estimated by the inflow water quality estimation circuitry and the water quality measurement value of the treated water acquired by the second water quality measurement value acquisition circuitry.

13. The aeration amount control device according to claim 10, further comprising a second water quality measurement value acquisition circuitry, wherein
the second water quality measurement value acquisition circuitry acquires a water quality measurement value of treated water that has been treated in the reaction tank, and
the target aeration amount calculation circuitry calculates the target aeration amount of aeration to be supplied from the blower to the reaction tank, on the basis of the inflow water quality value estimated by the inflow water quality estimation circuitry and the water quality measurement value of the treated water acquired by the second water quality measurement value acquisition circuitry.
